(19) United States
(12) Patent Application Publication
(10) Pub. No.: US 2023/0193498 A1
(43) Pub. Date: Jun. 22, 2023

(54) THREE-DIMENSIONAL CHITOSAN/SILVER COMPOSITE SCAFFOLD AND PREPARATION METHOD THEREOF

(71) Applicant: National Yang Ming Chiao Tung University, Hsinchu City (TW)

(72) Inventors: Pu-Wei WU, Zhubei City (TW); Bo-Han HUANG, Keelung City (TW); Yu-Jie CHIOU, Zhunan Township (TW); Li-Jie CHEN, Puyan Township (TW)

(21) Appl. No.: 17/837,002

(22) Filed: Jun. 9, 2022

(30) Foreign Application Priority Data
Dec. 16, 2021 (TW) 110147199

(51) Int. Cl. C25D 3/46; C25D 7/00; C25D 9/04; C25D 9/02

(52) U.S. Cl. CPC C25D 3/46; C25D 7/00; C25D 9/04; C25D 9/02; A61L 15/28

(57) ABSTRACT

A method for preparing a three-dimensional chitosan/silver composite scaffold includes mixing an acidic aqueous chitosan solution including protonated chitosan and a deposition accelerating agent being a soluble silver salt, spacedly disposing a cathode and an anode in the resultant suspension, and applying an electric field to the cathode and the anode so that the suspension undergoes electrodeposition. The suspension has a protonated chitosan concentration ranging from 0.7 to 2.8 w/v %, and a molarity of silver ions ranging from 4 to 60 mM. The composite scaffold prepared has columnar through-holes extending in a same extension direction and each having opposite first and second openings with the latter not less in width.

### CROSS-REFERENCE TO RELATED APPLICATION

This application claims priority of Taiwanese Patent Application No. 110147199, filed on Dec. 16, 2021.

### FIELD

The disclosure relates to a three-dimensional composite scaffold and a preparation method thereof, and more particularly to a three-dimensional chitosan/silver composite scaffold and a preparation method thereof.

### BACKGROUND

Chitosan is derivable from chitin which is the second most abundant natural polymer, and hence has a relatively low production cost. Furthermore, since chitosan has good biocompatibility (e.g. cyto-compatibility, etc.) and antibacterial activity, chitosan can be widely applied in the biomedical field as, for example, a dressing and an adsorption material.

Conventional three-dimensional chitosan scaffolds are generally classified into the following two categories: sponge-like chitosan scaffolds and net-like chitosan scaffolds.

The sponge-like chitosan scaffolds have an irregular shape, a non-uniform size, and pores that are distributed in a disordered manner and in communication with one another. Such chitosan scaffolds are prepared through a freeze-drying process or a salt-leaching process.

The freeze-drying process involves a phase transition of water, to be exact, direct sublimation from solid ice crystals to water vapor, for pore formation, such that the freeze-drying process is time-consuming and can hardly control the size and orientation of pores. Moreover, the three-dimensional chitosan scaffolds prepared through the freeze-drying process have a weak structure, and usually rely on subsequent processing to reinforce the structure thereof.

The salt-leaching process involves different solubility of salts in an aqueous chitosan solution at different temperatures. Specifically, many salt precipitates are formed at a low temperature, and such precipitates are washed off using pure water, such that pores are formed at the locations where salt precipitates are previously formed. However, since the pore formation attributed to the salt-leaching process totally relies on the particle size or distribution of the salt precipitates, the size and distribution of the pores can be hardly controlled, not to mention that it is difficult to have the pores oriented in a desired manner. Furthermore, there might be residual salt precipitates.

Since the pores of the sponge-like chitosan scaffolds are in communication with one another and have no desired orientation, the sponge-like chitosan scaffolds, when used in delivery of substances (such as drugs and/or cells), cannot deliver the substances in a desired direction, might deliver the substances slowly, and might lose the substances during the delivery. Therefore, the sponge-like chitosan scaffolds have limitations when applied as a material for drug delivery and/or cell growth (e.g. a drug delivery scaffold, a wound dressing, etc.).

Turning to the net-like chitosan scaffolds, such scaffolds have net-like layers stacked onto one another and pores that are uniform in size and distributed orderly and in a grid arrangement. Since such chitosan scaffolds are prepared through 3D (three-dimensional) printing in a predetermined manner, the size, shape, distribution, and orientation of the pores thereof can be controlled. However, the pores of the net-like chitosan scaffolds in the grid arrangement are in communication with one another, such that the net-like chitosan scaffolds, when used as a material for drug delivery and/or cell growth, cannot deliver drugs and cells in a desired direction.

Apart from the foregoing, conventional two-dimensional chitosan films may be prepared through an electrochemical process. In the electrochemical process, a voltage is applied to an acidic aqueous chitosan solution containing protonated chitosan, so that electrolysis of water occurs at the cathode to generate hydroxide ions and hydrogen gas bubbles. The hydroxide ions render the protonated chitosan deprotonated, so that neutral chitosan thus obtained is deposited on the surface of the cathode to form a chitosan film. Meanwhile, the hydrogen gas bubbles generated at the cathode cause the chitosan film to form many pores that have various sizes and shapes and are distributed in a disordered manner. Since excessive hydrogen gas bubbles are very likely to be generated during the electrochemical process and hence intervene the formation of the chitosan film, the chitosan film might have an insufficient thickness and an unsatisfactory structure strength. Accordingly, as reported in Huang et al. (2021), Carbohydrate Polymers, 273(12): 118560, polyethylene glycol and hydrogen peroxide may be added into an acidic aqueous solution containing protonated chitosan to inhibit generation of hydrogen gas bubbles so that the chitosan film deposited on the surface of the cathode is prevented from forming pores and has a smooth surface and a two-dimensional non-porous structure.

However, there is still a need to develop a satisfactory method for preparing a three-dimensional chitosan-based scaffold.

### SUMMARY

Therefore, a first object of the disclosure is to provide a method for preparing a three-dimensional chitosan/silver composite scaffold, which can alleviate at least one of the drawbacks of the prior art. The method includes:
mixing an acidic aqueous chitosan solution and a deposition accelerating agent to form a suspension, the acidic aqueous chitosan solution including protonated chitosan, the deposition accelerating agent being a soluble silver salt, the suspension having a concentration of the protonated chitosan which ranges from 0.7 w/v % to 2.8 w/v %, and a molarity of silver ions from the soluble silver salt which ranges from 4 mM to 60 mM;
spacedly disposing a cathode and an anode in the suspension; and
applying an electric field to the cathode and the anode so that the suspension undergoes electrodeposition, at the cathode, the silver ions in the suspension undergoing reduction to form silver metal, and water in the suspension undergoing electrolysis to form hydroxide ions and hydrogen gas bubbles, some of the hydroxide ions reacting with the silver ions to form silver oxide, and some other of the hydroxide ions deprotonating the protonated chitosan to obtain neutral chitosan, the silver oxide and the neutral chitosan being gradually deposited at a surface of the cathode in a direction toward the anode, the hydrogen gas bubbles growing in size from the cathode toward the anode, a three-dimensional chitosan/silver composite scaffold being formed on the surface of the cathode,
wherein the three-dimensional chitosan/silver composite scaffold includes a plurality of columnar through-holes which extend in a same extension direction, each of the columnar through-holes having a first opening and a second opening opposite to the first opening in the extension direction and larger in width than the first opening.

A second object of the disclosure is to provide a three-dimensional chitosan/silver composite scaffold which can alleviate at least one of the drawbacks of the prior art. The three-dimensional chitosan/silver composite scaffold includes:
a main body made from chitosan, silver oxide, and silver metal; and
a plurality of columnar through-holes extending through the main body in a same extension direction, each of the columnar through-holes having a first opening and a second opening that is opposite to the first opening in the extension direction and that is not less in width than the first opening.

### BRIEF DESCRIPTION OF THE DRAWINGS

Other features and advantages of the disclosure will become apparent in the following detailed description of the embodiment(s) with reference to the accompanying drawings, of which:

FIG. 1 is a scanning electron microscope (SEM) image showing a section of the three-dimensional chitosan/silver composite scaffold of Example 1 according to the present disclosure;

FIG. 2 is an SEM image showing an initially deposited surface of the three-dimensional chitosan/silver composite scaffold of Example 1;

FIG. 3 is an SEM image showing a lastly deposited surface of the three-dimensional chitosan/silver composite scaffold of Example 1;

FIG. 4 is an X-ray diffractogram of the three-dimensional chitosan/silver composite scaffold of Example 1;

FIG. 5 shows results of X-ray photoemission spectroscopy (XPS) of the three-dimensional chitosan/silver composite scaffold of Example 1, in which the left portion shows the silver content and silver ion content in the initially deposited surface (represented by the symbol “A”), lastly deposited surface (represented by the symbol “E”), portions therebetween (respectively represented by the symbols “B”, “C”, and “D”) of the three-dimensional chitosan/silver composite scaffold of Example 1, as well as an SEM image showing a section of the three-dimensional chitosan/silver composite scaffold of Example 1, and the right portion shows X-ray photoemission spectra of the initially deposited surface, lastly deposited surface, portions therebetween of the three-dimensional chitosan/silver composite scaffold of Example 1;

FIG. 6 is an SEM image showing a section of the three-dimensional chitosan/silver composite scaffold of Example 2 according to the present disclosure;

FIG. 7 is an SEM image showing a lastly deposited surface of the three-dimensional chitosan/silver composite scaffold of Example 2;

FIG. 8 is an SEM image showing a section of the three-dimensional chitosan/silver composite scaffold of Example 3 according to the present disclosure;

FIG. 9 is an SEM image showing a lastly deposited surface of the three-dimensional chitosan/silver composite scaffold of Example 3;

FIG. 10 is an SEM image showing a section of the three-dimensional chitosan/silver composite scaffold of Example 4 according to the present disclosure;

FIG. 11 is an SEM image showing a lastly deposited surface of the three-dimensional chitosan/silver composite scaffold of Example 4;

FIG. 12 is an SEM image showing a section of the three-dimensional chitosan/silver composite scaffold of Example 5 according to the present disclosure;

FIG. 13 is an SEM image showing a lastly deposited surface of the three-dimensional chitosan/silver composite scaffold of Example 5;

FIG. 14 is an SEM image showing a section of the three-dimensional chitosan/silver composite scaffold of Example 6 according to the present disclosure;

FIG. 15 is an SEM image showing a section and an initially deposited surface of the three-dimensional chitosan/silver composite scaffold of Example 6;

FIG. 16 is an SEM image showing a lastly deposited surface of the three-dimensional chitosan/silver composite scaffold of Example 6;

FIG. 17 is an SEM image showing a section of the chitosan film of Comparative Example 1;

FIG. 18 is an SEM image showing a surface of the chitosan film of Comparative Example 1;

FIG. 19 is an SEM image showing a section of the chitosan film of Comparative Example 2; and

FIG. 20 is an SEM image showing a surface of the chitosan film of Comparative Example 2.

### DETAILED DESCRIPTION

As used herein, the concentration expressed with the unit “w/v %” is defined as a weight (in g) of a solute divided by a total volume (in mL) of a solution containing the solute.

As used herein, the molarity expressed with the unit “mM” is defined as a millimole (mmol) of a solute divided by a total volume (in L) of a solution containing the solute.

The present disclosure provides a method for preparing a three-dimensional chitosan/silver composite scaffold, which includes the following steps.

An acidic aqueous chitosan solution and a deposition accelerating agent are mixed to form a suspension. The acidic aqueous chitosan solution includes protonated chitosan (also referred to as positively charged chitosan). The deposition accelerating agent is a soluble silver salt. The suspension has a concentration of the protonated chitosan which ranges from 0.7 w/v% to 2.8 w/v %, and a molarity of silver ions formed from the soluble silver salt which ranges from 4 mM to 60 mM.

A cathode and an anode are disposed to be spaced apart from each other in the suspension.

An electric field is applied to the cathode and the anode so that the suspension undergoes electrodeposition. At the cathode, the silver ions in the suspension undergo reduction to form silver metal, and water in the suspension undergoes electrolysis to form hydroxide ions and hydrogen gas bubbles. Some of the hydroxide ions react with the silver ions to form silver oxide, and some other of the hydroxide ions contact with and deprotonate the protonated chitosan to form neutral chitosan. The silver oxide and the neutral chitosan, which are continuously generated, are gradually deposited at a surface of the cathode in a direction toward the anode (namely, the thickness of the deposited main body gradually increases in the direction toward the anode). The hydrogen gas bubbles, which are continuously generated at the cathode, grow in size from the cathode toward the anode, and enable formation of columnar through-holes in the deposited main body. Accordingly, a three-dimensional chitosan/silver composite scaffold including the main body and the columnar through-holes is formed on the surface of the cathode. The detail of such scaffold is described later after describing the detail of the preparation method.

Preparation of the acidic aqueous chitosan solution is not limited to any preparation process, as long as the protonated chitosan can be formed. For instance, chitosan may be dissolved in an acidic aqueous solution having a pH value ranging from 0 to 6.5 to be protonated, so as to prepare the acidic aqueous chitosan solution including the protonated chitosan. The acidic aqueous solution may be prepared by mixing at least one of acetic acid and hydrochloric acid with water.

The soluble silver salt (i.e. the deposition accelerating agent) may be a water-soluble silver salt. Anions formed from the soluble silver salt may not undergo physical interaction and/or chemical interaction with the protonated chitosan in the suspension so that the suspension can be prevented from gelation. The soluble silver salt may be selected from the group consisting of silver nitrate, silver acetate, silver carbonate, and combinations thereof.

The following should be noted. When the concentration of the protonated chitosan in the suspension is less than 0.7 w/v %, the silver oxide and the chitosan are deposited in low efficiency, and the hydrogen gas bubbles hence cannot be guided to grow in size only in a direction from the cathode to the anode and be elongated in such direction for the columnar through-holes to be formed, such that the three-dimensional chitosan/silver composite scaffold cannot be prepared. When the concentration of the protonated chitosan in the suspension is greater than 2.8 w/v %, the suspension might undergo gelation, adversely affecting the progression of the electrodeposition. When the molarity of the silver ions in the suspension is less than 4 mM, the silver oxide and the chitosan are deposited in low efficiency, and the hydrogen gas bubbles hence cannot be guided to grow in size only in a direction from the cathode to the anode and be elongated in such direction for the columnar through-holes to be formed, such that the three-dimensional chitosan/silver composite scaffold cannot be prepared. When the molarity of the silver ions in the suspension is greater than 60 mM, the suspension might undergo gelation, adversely affecting the progression of the electrodeposition.

In certain embodiments, the concentration of the protonated chitosan in the suspension ranges from 1 w/v % to 2 w/v %, and the molarity of the silver ions in the suspension ranges from 12 mM to 30 mM.

In certain embodiments, the acidic aqueous chitosan solution and the deposition accelerating agent are mixed further with a stabilizing agent for slowing the electrolysis of the water so as to form the suspension. Since the stabilizing agent does not undergo a redox reaction, addition of such agent can lower the percentage of water in the suspension, reducing the electrolysis of water in a unit of time and hence lowering the speed of generation of the hydrogen gas bubbles. However, it should be noted that the stabilizing agent is not a necessary ingredient for preparing the three-dimensional chitosan/silver composite scaffold. Namely, the three-dimensional chitosan/silver composite scaffold can still be formed without using the stabilizing agent. The stabilizing agent may be selected from the group consisting of polyethylene glycol, methanol, ethanol, isopropanol, and combinations thereof. A concentration of the stabilizing agent in the suspension may be greater than 0 w/v % and not greater than 40 w/v %. When the concentration of the stabilizing in the suspension is not greater than 40 w/v %, the suspension can be prevented from gelation. When the stabilizing agent is polyethylene glycol, it is not necessary to particularly limit the molecular weight of polyethylene glycol, as long as the molecular weight of polyethylene glycol can be adjusted according to the concentration of polyethylene glycol in the suspension to prevent the suspension from gelation. The molecular weight of polyethylene glycol may range from 400 Da to 20,000 Da.

Regarding the electrodeposition, as long as the molarity of the silver ions (from the soluble silver salt) in the suspension ranges from 4 mM to 60 mM and the concentration of the protonated chitosan in the suspension ranges from 0.7 w/v % to 2.8 w/v %, the deposition of the silver oxide and chitosan is similar in speed to the generation of the hydrogen gas bubbles so that the hydrogen gas bubbles can be guided to grow in size only in a direction from the cathode to the anode in a steady manner and be elongated in such direction for the columnar through-holes to be formed and for not intervening the growth of the scaffold on the surface of the cathode. Thus, it is not necessary to particularly limit the operation conditions of the electrodeposition. For example, the electric field may range from 1 V/cm to 10 V/cm, and the time for the electrodeposition may range from 1 minute to 60 minutes.

The preparation method of the present disclosure may further comprise adding into the suspension a hydrogen gas inhibitor for reducing an amount of the hydrogen gas bubbles. In other words, the suspension may undergo the electrodeposition in the presence of the hydrogen gas inhibitor. The hydrogen gas inhibitor is able to react with the hydrogen gas bubbles generated during the electrodeposition to form water, and hence can prevent presence of excessive hydrogen gas bubbles during the electrodeposition. However, it should be noted that the hydrogen gas inhibitor is not a necessary ingredient for preparing the three-dimensional chitosan/silver composite scaffold. Namely, the three-dimensional chitosan/silver composite scaffold can still be formed without using the hydrogen gas inhibitor.

The hydrogen gas inhibitor may be hydrogen peroxide. A concentration of the hydrogen gas inhibitor in the suspension may be greater than 0 w/v % and not greater than 11 w/v %. The term “hydrogen peroxide” may refer to pure hydrogen peroxide or an aqueous hydrogen peroxide solution (prepared by dissolving pure hydrogen peroxide in water). Since hydrogen peroxide is usually stored in the form of an aqueous hydrogen peroxide solution, the concentration of the hydrogen gas inhibitor in the suspension can be adjusted to a desired value based on the concentration of hydrogen peroxide in the aqueous hydrogen peroxide solution. For instance, when a 30% aqueous hydrogen peroxide solution (i.e. such solution has 70% of water) is used as the hydrogen gas inhibitor, the concentration of the hydrogen gas inhibitor in the suspension can be adjusted to not greater than 11 w/v % based on the concentration of hydrogen peroxide in the aqueous hydrogen peroxide solution, so that the concentration of the protonated chitosan and the molarity of the silver ions in the suspension can be prevented from excessive dilution.

After completion of the electrodeposition, the three-dimensional chitosan/silver composite scaffold can be peeled off from the surface of the cathode. For subsequent analysis or application, the three-dimensional chitosan/silver composite scaffold may be subjected to freeze-drying, thermal drying, or natural drying.

The three-dimensional chitosan/silver composite scaffold of the present disclosure includes the main body and the columnar through-holes. The main body is made from chitosan, silver oxide, and silver metal. The columnar through-holes extend through the main body in a same extension direction. Each of the columnar through-holes has a first opening and a second opening that is opposite to the first opening in the extension direction and larger in width than the first opening.

When the suspension for preparing the three-dimensional chitosan/silver composite scaffold includes the stabilizing agent, the main body of the three-dimensional chitosan/silver composite scaffold is made further from the stabilizing agent.

The first openings of the columnar through-holes of the three-dimensional chitosan/silver composite scaffold are formed on a surface of the three-dimensional chitosan/silver composite scaffold in contact with the cathode. The first openings of the columnar through-holes may have a width ranging from 60 μm to 1,000 μm. The second openings of the columnar through-holes may have a width ranging from 200 μm to 1,000 μm. Each of the columnar through-holes has a depth and a ratio of the first opening to the depth that may range from 1:1 to 1:35.

The present disclosure will be further described by way of the following examples. However, it should be understood that the following examples are intended solely for the purpose of illustration and should not be construed as limiting the present disclosure in practice.

### Three-Dimensional Chitosan/Silver Composite Scaffold of Example 1 According to Present Disclosure

### Preparation

1 g of chitosan powder was added into 50 cc of an aqueous acetic acid solution (having a pH value of 4.6), followed by evenly stirring at 70° C., so that the chitosan powder was dissolved and protonated to form an acidic aqueous chitosan solution containing protonated chitosan. Subsequently, silver nitrate solution (i.e., dissolved in water) and polyethylene glycol (having a molecular weight of 600 Da) were added into the acidic aqueous chitosan solution, followed by stirring for 24 hours, so that a suspension containing silver ions (the molarity of which was 20 mM), protonated chitosan (the concentration of which was 1.4 w/v %), and polyethylene glycol (the concentration of which was 10 w/v %) was formed. 50 cc of the suspension was placed in an electrolytic tank, followed by adding 1.1 g of an aqueous hydrogen peroxide solution (having a hydrogen peroxide concentration of 30 wt %) into the suspension and evenly stirring. A cathode (which was a stainless steel plate having an area of 17.5 cm²) and an anode (which was a platinum plate having an area of 17.5 cm²) were spacedly disposed in the electrolytic tank to obtain an electrolytic cell. An electric field of 3.3 V/cm was then applied to the electrolytic cell for electrodeposition to proceed for 10 minutes. Therefore, the three-dimensional chitosan/silver composite scaffold of Example 1 according to the present disclosure was formed on the surface of the cathode and had columnar through-holes.

### Analysis

The three-dimensional chitosan/silver composite scaffold of Example 1 was peeled off from the surface of the cathode, and was then placed in a refrigerator to be subjected to a freezing treatment at −80° C. for 48 hours. Subsequently, the three-dimensional chitosan/silver composite scaffold of Example 1 was placed in a freeze dryer to be subjected to a low-temperature vacuum drying treatment for 72 hours.

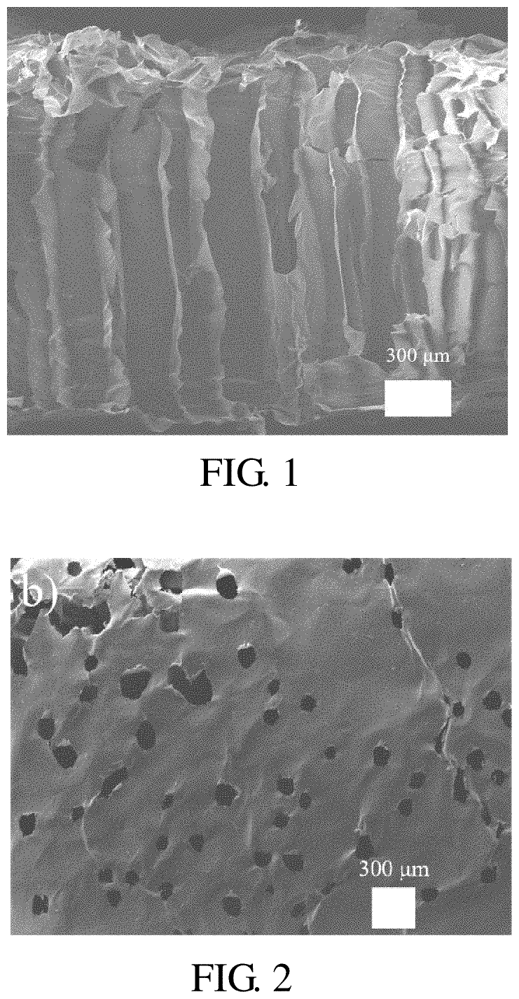

Afterward, the three-dimensional chitosan/silver composite scaffold of Example 1, which had been dried, was subjected to morphological analysis using a scanning electron microscope (SEM) (manufacturer: JEOL; model: JSM-6700F). The SEM images thus obtained are shown in FIGS. 1 to 3.

Furthermore, the three-dimensional chitosan/silver composite scaffold of Example 1, which had been dried, was subjected to qualitative analysis using an X-ray diffractometer (manufacturer: Bruker; model: D2 PHASER). The X-ray diffractogram thus obtained is shown in FIG. 4.

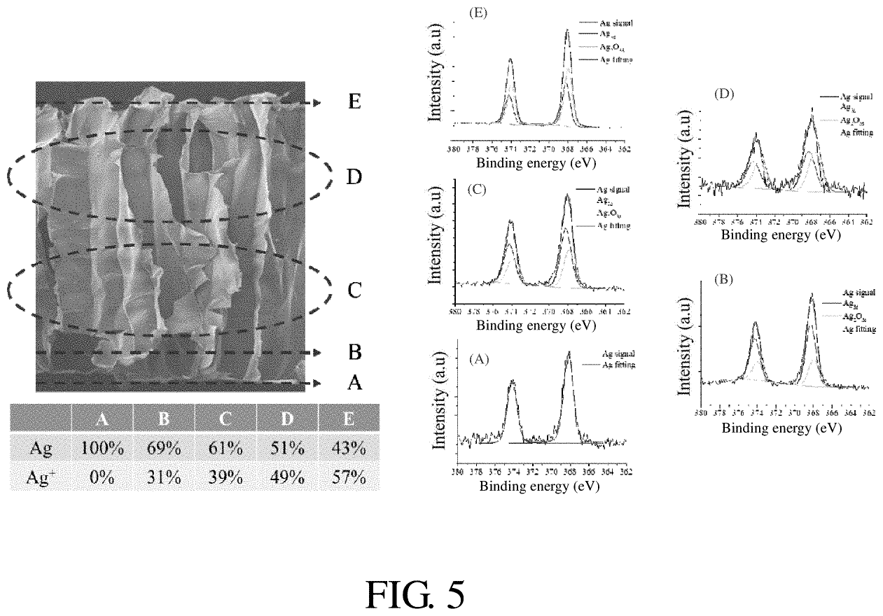

In addition, the three-dimensional chitosan/silver composite scaffold of Example 1, which had been dried, was subjected to compositional analysis using an X-ray photoemission spectrometer (manufacturer: Thermo Fisher Scientific; model: ESCALAB Xi+). The X-ray photoemission spectra thus obtained and the results of the compositional analysis calculated from such spectra are shown in FIG. 5.

It should be noted that the aforesaid freezing treatment and low-temperature vacuum drying treatment were only conducted for the analysis mentioned above, and were not necessary steps to prepare the three-dimensional chitosan/silver composite scaffold of the present disclosure.

### Results and Discussion

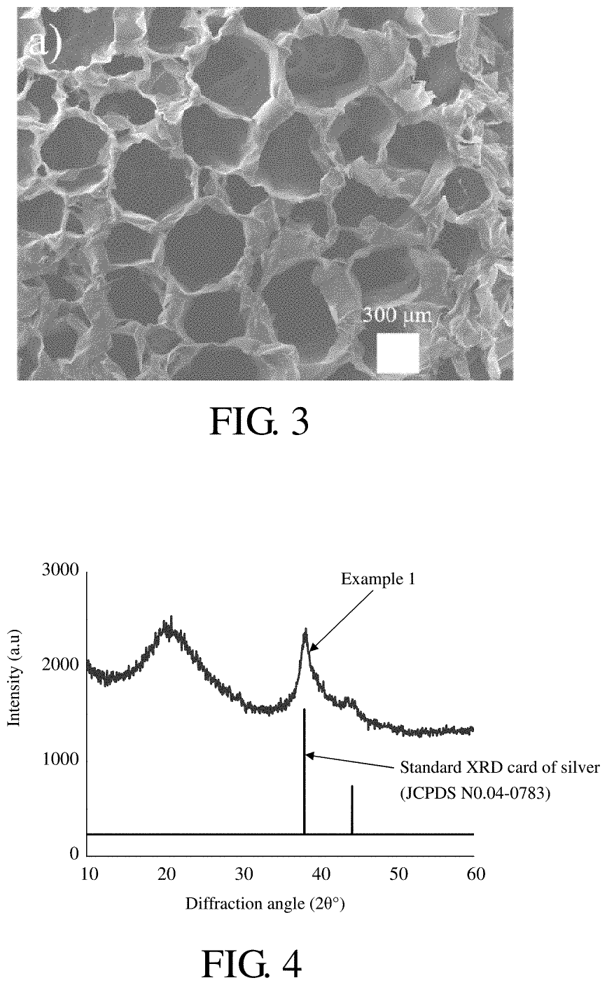

FIG. 1 shows a cross-sectional view of the three-dimensional chitosan/silver composite scaffold of Example 1. FIG. 2 shows the surface of the three-dimensional chitosan/silver composite scaffold of Example 1 in contact with the cathode, which was the surface initially formed during the electrodeposition (such surface is referred to as “initially deposited surface” hereinafter). FIG. 3 shows the surface of the three-dimensional chitosan/silver composite scaffold of Example 1 away from the cathode, which was the surface lastly formed during the electrodeposition (such surface is referred to as “lastly deposited surface” hereinafter).

As shown in FIG. 1, the three-dimensional chitosan/silver composite scaffold of Example 1 had a thickness approximately ranging from 1,500 μm to 2,000 μm, as well as columnar through-holes extending in the same extension direction. As shown in FIGS. 2 and 3, the columnar through-holes had smaller openings (having a width approximately ranging from 60 μm to 230 μm) on the initially deposited surface and larger openings (having a width approximately ranging from 130 μm to 660 μm) on the lastly deposited surface. If the openings on the initially deposited surface are defined as “first opening”, and if the openings on the lastly deposited surface are defined as “second opening”, based on FIGS. 2 and 3, it can be verified that each of the columnar through-holes of the three-dimensional chitosan/silver composite scaffold according to the present disclosure has a first opening and a second opening which is opposite to the first opening in the extension direction and larger in width than the first opening.

Referring to FIG. 4, the three-dimensional chitosan/silver composite scaffold of Example 1 had a signal peak at 2θ=20.2° indicating the a phase crystal structure of chitosan, as well as signal peaks at 2θ=38.1° and 2θ=44.3° respectively indicating the (111) and (200) crystal planes.

Referring to FIG. 5, the symbol “A” represents the initially deposited surface of the three-dimensional chitosan/silver composite scaffold of Example 1, the symbol represents the lastly deposited surface of the three-dimensional chitosan/silver composite scaffold of Example 1, and the symbols “B”, “C”, and “D” respectively represent the portions of the three-dimensional chitosan/silver composite scaffold of Example 1 between the initially deposited surface and the lastly deposited surface. As shown in FIG. 5, the three-dimensional chitosan/silver composite scaffold of Example 1 contained silver metal and silver oxide. Furthermore, in the direction from A to E (i.e. in the direction from the initially deposited surface to the lastly deposited surface), the three-dimensional chitosan/silver composite scaffold of Example 1 had a silver content gradually decreasing from 100% to 43%, as well as a content of silver ions from silver oxide which gradually increased from 0% to 57%.

### Three-Dimensional Chitosan/Silver Composite Scaffolds of Examples 2 to 5 According to Present Disclosure

### Preparation

The three-dimensional chitosan/silver composite scaffolds of Examples 2 and 3 were prepared using a procedure similar to that for the three-dimensional chitosan/silver composite scaffold of Example 1, except that the soluble silver salt for preparing the suspension was modified. Specifically, the soluble silver salt for preparing Example 2 was silver carbonate, and that for preparing Example 3 was silver acetate.

The three-dimensional chitosan/silver composite scaffolds of Examples 4 and 5 were prepared using a procedure similar to that for the three-dimensional chitosan/silver composite scaffold of Example 1, except that the protonated chitosan concentration and the molarity of silver ions in the suspension were modified. Specifically, the protonated chitosan concentration and the molarity of silver ions in the suspension for preparing Example 4 were 2.8 w/v % and 4 mM, respectively, and the protonated chitosan concentration and the molarity of silver ions in the suspension for preparing Example 5 were 0.7 w/v % and 60 mM, respectively.

### Analysis

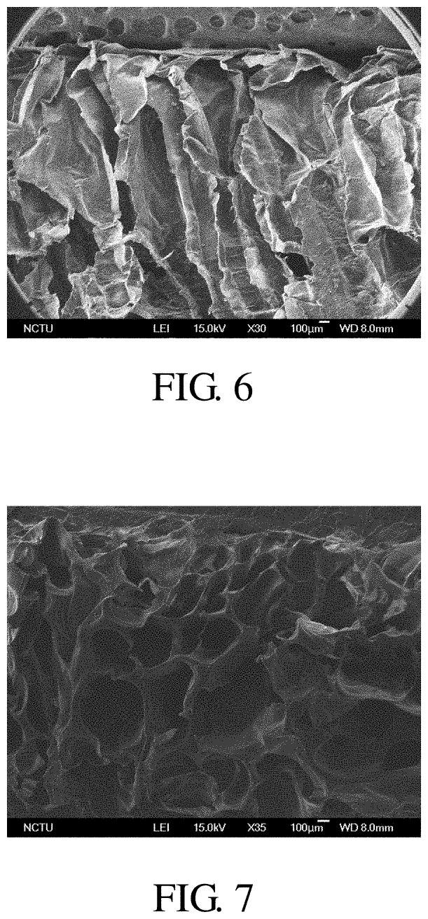

The three-dimensional chitosan/silver composite scaffolds of Examples 2 to 5 were subjected to morphological analysis using the same procedure for the three-dimensional chitosan/silver composite scaffold of Example 1. The SEM images thus obtained are shown in FIGS. 6 to 13.

### Results and Discussion

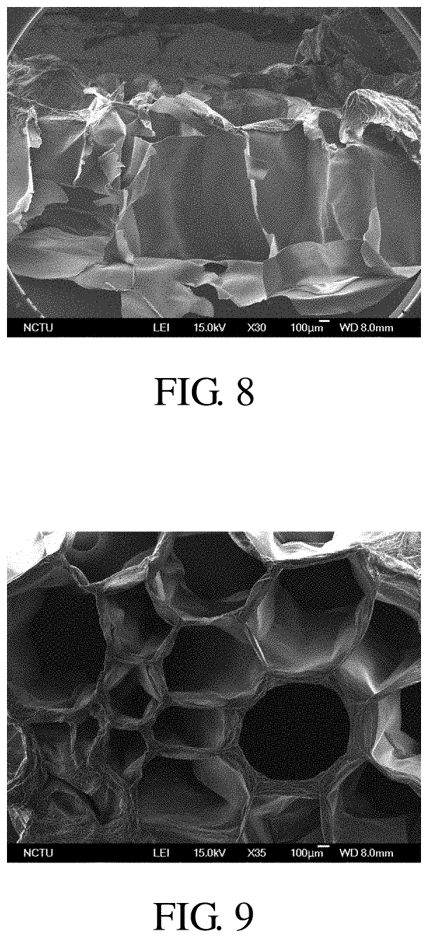

FIG. 6 shows a cross-sectional view of the three-dimensional chitosan/silver composite scaffold of Example 2, and FIG. 7 shows a lastly deposited surface of the three-dimensional chitosan/silver composite scaffold of Example 2. FIG. 8 shows a cross-sectional of the three-dimensional chitosan/silver composite scaffold of Example 3, and FIG. 9 shows a lastly deposited surface of the three-dimensional chitosan/silver composite scaffold of Example 3. As shown in FIGS. 6 to 9, the three-dimensional chitosan/silver composite scaffolds of Examples 2 and 3, which were respectively prepared using silver carbonate and silver acetate, both had columnar through-holes extending in the same extension direction.

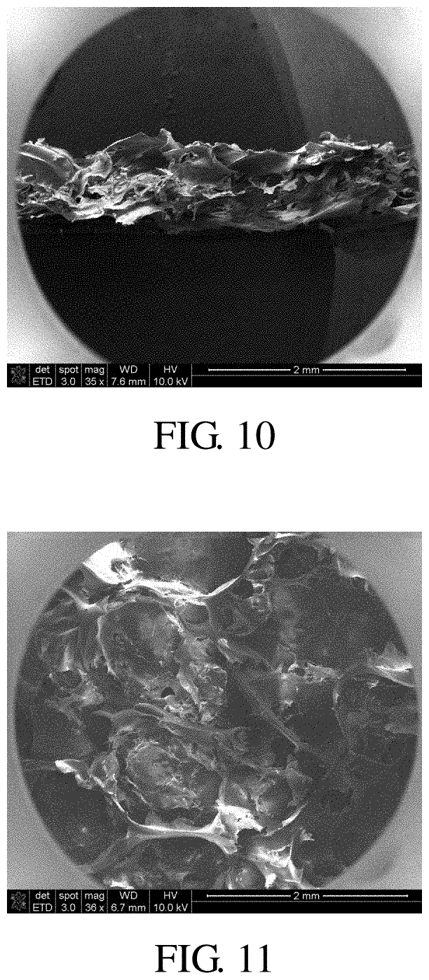

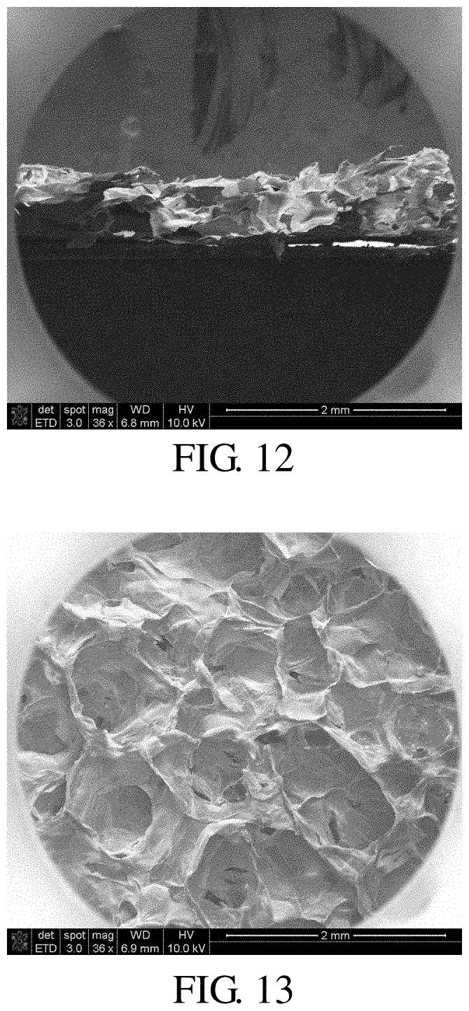

FIG. 10 shows a cross-sectional view of the three-dimensional chitosan/silver composite scaffold of Example 4, and FIG. 11 shows a lastly deposited surface of the three-dimensional chitosan/silver composite scaffold of Example 4. FIG. 12 shows a cross-sectional view of the three-dimensional chitosan/silver composite scaffold of Example 5, and FIG. 13 shows a lastly deposited surface of the three-dimensional chitosan/silver composite scaffold of Example 5. Based on FIGS. 10 to 13, it can be inferred that columnar through-holes had begun to be formed in the three-dimensional chitosan/silver composite scaffolds of Examples 4 and 5, and it can be observed that openings were formed on the lastly deposited surfaces of the three-dimensional chitosan/silver composite scaffolds of Examples 4 and 5. These results reveal that the suspension having the protonated chitosan concentration ranging from 0.7 w/v % to 2.8 w/v % and the molarity of silver ions ranging from 4 mM to 60 mM can be applied to prepare the three-dimensional chitosan/silver composite scaffold according to the present disclosure.

### Three-Dimensional Chitosan/Silver Composite Scaffold of Example 6 According to Present Disclosure

### Preparation

1 g of chitosan powder was added into 50 cc of an aqueous acetic acid solution (having a pH value of 4.6), followed by evenly stirring at 70° C., so that the chitosan powder was dissolved and protonated to form an acidic aqueous chitosan solution containing protonated chitosan. Subsequently, silver nitrate solution (dissolved in water) was added into the acidic aqueous chitosan solution, followed by stirring for 24 hours, so that a suspension containing silver ions (the molarity of which was 20 mM) and protonated chitosan (the concentration of which was 1.4 w/v %) was formed. 50 cc of the suspension was placed in an electrolytic tank. A cathode (which was a stainless steel plate having an area of 17.5 cm²) and an anode (which was a platinum plate having an area of 17.5 cm²) were spacedly disposed in the electrolytic tank to obtain an electrolytic cell. An electric field of 3.3 V/cm was then applied to the electrolytic cell for electrodeposition to proceed for 10 minutes. Therefore, the three-dimensional chitosan/silver composite scaffold of Example 6 according to the present disclosure was formed on the surface of the cathode and had columnar through-holes.

### Analysis

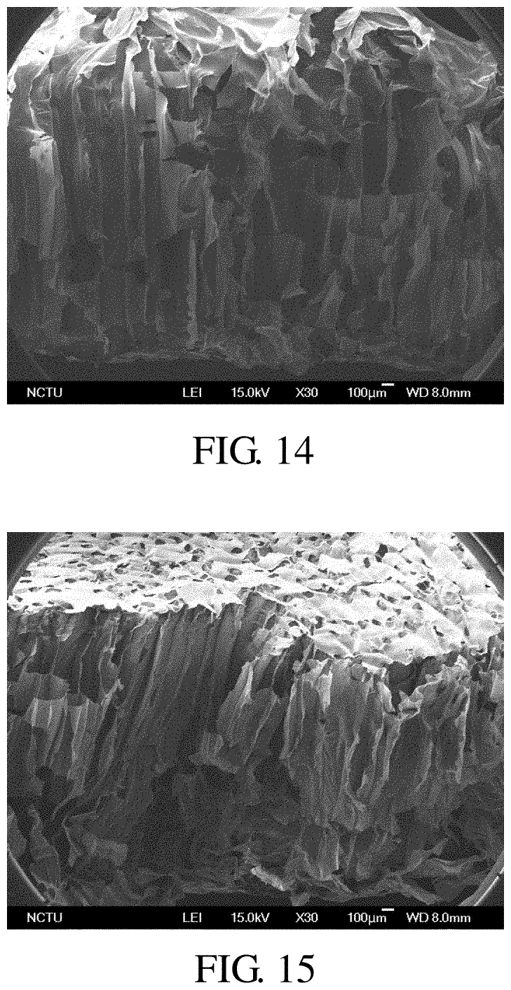

The three-dimensional chitosan/silver composite scaffold of Example 6 was subjected to morphological analysis using the same procedure for the three-dimensional chitosan/silver composite scaffold of Example 1. The SEM images thus obtained are shown in FIGS. 14 to 16.

### Results and Discussion

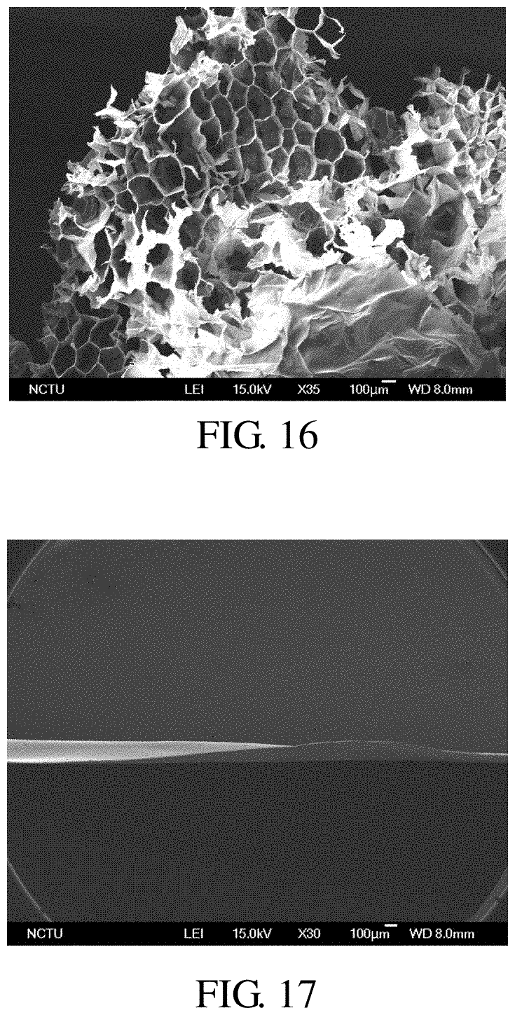

FIG. 14 shows a cross-sectional view of the three-dimensional chitosan/silver composite scaffold of Example 6, FIG. 15 shows a cross-sectional view and an initially deposited surface of the three-dimensional chitosan/silver composite scaffold of Example 6, and FIG. 16 shows a lastly deposited surface of the three-dimensional chitosan/silver composite scaffold of Example 6. It can be verified from FIGS. 14 to 16 that the three-dimensional chitosan/silver composite scaffold according to the present disclosure can be prepared in the absence of polyethylene glycol and hydrogen peroxide.

### Chitosan Films of Comparative Examples 1 and 2

### Preparation

The preparation procedure for the chitosan films of Comparative Examples 1 and 2 is different from that for the three-dimensional chitosan/silver composite scaffold of Example 1 in that another soluble metal salt was used instead of the soluble silver salt to prepare a suspension. Specifically, in preparing the chitosan film of Comparative Example 1, a soluble gold salt was used to prepare a suspension having a molarity of gold ions that was 4 mM. Moreover, in preparing the chitosan film of Comparative Example 2, a soluble copper salt was used to prepare a suspension having a molarity of copper ions that was 20 mM.

### Analysis

The chitosan films of Comparative Examples 1 and 2 were subjected to morphological analysis using the same procedure for the three-dimensional chitosan/silver composite scaffold of Example 1. The SEM images thus obtained are shown in FIGS. 17 to 20.

### Results and Discussion

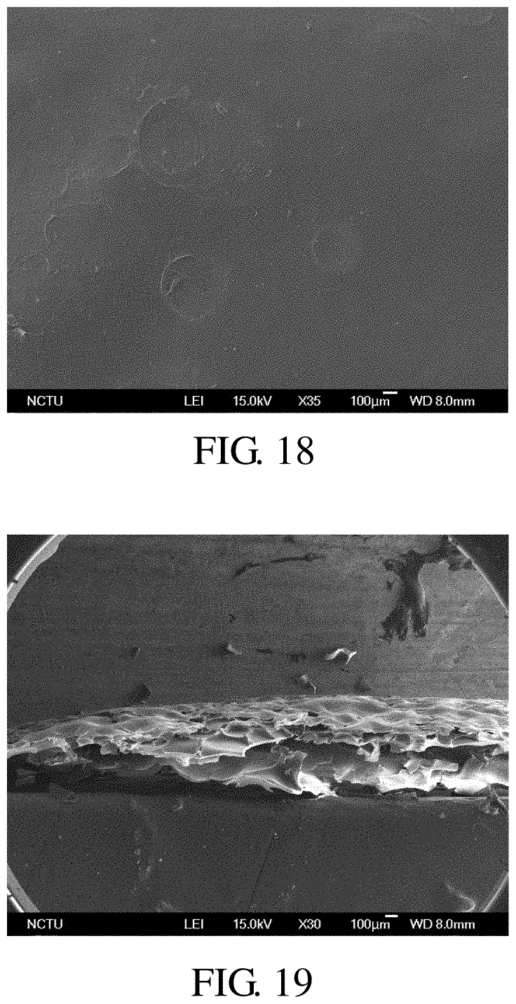

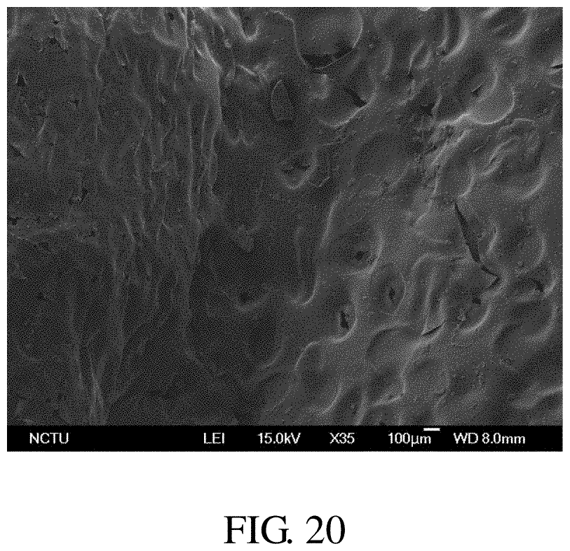

FIG. 17 shows a cross-sectional view of the chitosan film of Comparative Example 1, and FIG. 18 shows a surface of the chitosan film of Comparative Example 1. FIG. 19 shows a cross-sectional of the chitosan film of Comparative Example 2, and FIG. 20 shows a surface of the chitosan film of Comparative Example 2. It can be verified from FIGS. 17 to 20 that the soluble gold salt used for Comparative Example 1 and the soluble copper salt used for Comparative Example 2 can result in a two-dimensional chitosan film only, not a three-dimensional chitosan-based scaffold including columnar through-holes.

### Conclusion Based on Aforesaid Examples and Comparative Examples

In view of the results of Examples 1 to 6 and Comparative Examples 1 and 2, it can be proven that, by subjecting a suspension having a protonated chitosan concentration ranging from 0.7 w/v % to 2.8 w/v % and a molarity of silver ions ranging from 4 mM to 60 mM to electrodeposition, a three-dimensional chitosan/silver composite scaffold having columnar through-holes extending in the same extension direction can be prepared. Such three-dimensional chitosan/silver composite scaffold contains silver metal and silver oxide.

The advantages of the scaffold and method according to the present disclosure are described as follows.

By virtue of the molarity of the silver ions (from the soluble silver salt) ranging from 4 mM to 60 mM and the protonated chitosan concentration ranging from 0.7 w/v % to 2.8 w/v %, the deposition of the silver oxide and chitosan is similar in speed to the generation of the hydrogen gas bubbles, so the hydrogen gas bubbles can be guided to grow in size only in a direction from the cathode to the anode in a steady manner and be elongated in such direction for the columnar through-holes to be formed and for not intervening the growth of the scaffold. Thus, the columnar through-holes can be formed in the main body of the scaffold made from the silver oxide and chitosan, the thickness of which gradually increases in a direction toward the anode. Accordingly, the three-dimensional chitosan/silver composite scaffold of the present disclosure can be formed on the surface of the cathode.

Regarding the three-dimensional chitosan/silver composite scaffold of the present disclosure, the silver metal therein has antibacterial activity, and the chitosan therein has antibacterial activity, biocompatibility, and metal adsorbability. Furthermore, the three-dimensional chitosan/silver composite scaffold has the columnar through-holes extending in the same extension direction (i.e. having the same orientation), and hence can transport a substance (e.g. a drug, a cell, etc.) in a desired direction. Therefore, the three-dimensional chitosan/silver composite scaffold of the present disclosure is suitable to be used as, for example, an antibacterial dressing for drug delivery and/or cell growth, or a filter material having metal adsorbability and/or antibacterial activity.

In addition, the method of the present disclosure is simple and time-efficient, being suitable for mass production of the three-dimensional chitosan/silver composite scaffold. The ingredients used in the method of the present disclosure do not have biotoxicity and are environmentally friendly.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method for preparing a three-dimensional chitosan/silver composite scaffold, comprising:
mixing an acidic aqueous chitosan solution and a deposition accelerating agent to form a suspension, the acidic aqueous chitosan solution including protonated chitosan, the deposition accelerating agent being a soluble silver salt, the suspension having a concentration of the protonated chitosan which ranges from 0.7 w/v % to 2.8 w/v %, and a molarity of silver ions from the soluble silver salt which ranges from 4 mM to 60 mM;
spacedly disposing a cathode and an anode in the suspension; and
applying an electric field to the cathode and the anode so that the suspension undergoes electrodeposition, at the cathode, the silver ions in the suspension undergoing reduction to form silver metal, and water in the suspension undergoing electrolysis to form hydroxide ions and hydrogen gas bubbles, some of the hydroxide ions reacting with the silver ions to form silver oxide, and some other of the hydroxide ions deprotonating the protonated chitosan to form neutral chitosan, the silver oxide and the neutral chitosan being gradually deposited at a surface of the cathode in a direction toward the anode, the hydrogen gas bubbles growing in size from the cathode toward the anode, a three-dimensional chitosan/silver composite scaffold being formed on the surface of the cathode,
wherein the three-dimensional chitosan/silver composite scaffold includes a plurality of columnar through-holes which extend in a same extension direction, each of the columnar through-holes having a first opening and a second opening that is opposite to the first opening in the extension direction and that is not less in width than the first opening.

2. The method as claimed in claim 1, wherein the soluble silver salt is a water-soluble silver salt, anions from the soluble silver salt being free from interaction with the protonated chitosan in the suspension.

3. The method as claimed in claim 2, wherein the soluble silver salt is selected from the group consisting of silver nitrate, silver acetate, silver carbonate, and combinations thereof.

4. The method as claimed in claim 1, wherein the acidic aqueous chitosan solution and the deposition accelerating agent are mixed further with a stabilizing agent for slowing the electrolysis of the water so as to form the suspension, a concentration of the stabilizing agent in the suspension being greater than 0 w/v % and not greater than 40 w/v %.

5. The method as claimed in claim 4, wherein the stabilizing agent is selected from the group consisting of polyethylene glycol, methanol, ethanol, isopropanol, and combinations thereof.

6. The method as claimed in claim 1, wherein the acidic aqueous chitosan solution is obtainable by dissolving chitosan in an acidic aqueous solution having a pH value ranging from 0 to 6.5.

7. The method as claimed in claim 1, further comprising adding into the suspension a hydrogen gas inhibitor for reducing an amount of the hydrogen gas bubbles, the suspension undergoing the electrodeposition in the presence of the hydrogen gas inhibitor.

8. The method as claimed in claim 7, wherein the hydrogen gas inhibitor is hydrogen peroxide, a concentration of the hydrogen gas inhibitor in the suspension being greater than 0 w/v % and not greater than 11 w/v %.

9. The method as claimed in claim 1, wherein the first openings of the columnar through-holes of the three-dimensional chitosan/silver composite scaffold are formed on a surface of the three-dimensional chitosan/silver composite scaffold in contact with the cathode, the first openings of the columnar through-holes having a width ranging from 60 μm to 1,000 μm, the second openings of the columnar through-holes having a width ranging from 200 μm to 1,000 μm, each of the columnar through-holes having a depth and a ratio of the first opening to the depth that ranges from 1:1 to 1:35.

10. A three-dimensional chitosan/silver composite scaffold, comprising:
a main body made from chitosan, silver oxide, and silver metal; and
a plurality of columnar through-holes extending through the main body in a same extension direction, each of the columnar through-holes having a first opening and a second opening that is opposite to the first opening in the extension direction and that is not less in width than the first opening.

11. The three-dimensional chitosan/silver composite scaffold as claimed in claim 10, wherein the first openings of the columnar through-holes have a width ranging from 60 μm to 1,000 μm, and the second openings of the columnar through-holes have a width ranging from 200 μm to 1,000 μm.

12. The three-dimensional chitosan/silver composite scaffold as claimed in claim 10, wherein each of the columnar through-holes has a depth and a ratio of the first opening to the depth that ranges from 1:1 to 1:35.
